(19) United States
(12) Patent Application Publication
(10) Pub. No.: US 2011/0283820 A1
(43) Pub. Date: Nov. 24, 2011

(54) MULTI-SENSOR HEAD

(75) Inventor: Josef Glaser, Graz (AT)

(73) Assignee: KISTLER HOLDING AG, WINTERTHUR (CH)

(21) Appl. No.: 13/146,576

(22) PCT Filed: Mar. 4, 2010
(86) PCT No.: PCT/CH10/00055

§ 371 Date: Jul. 27, 2011

(30) Foreign Application Priority Data
Mar. 16, 2009 (CH) CH 392/09

(51) Int. Cl. G01N 33/00

(52) U.S. Cl. 73/865.8

(57) ABSTRACT

A multi-sensor head for installation in an installation opening of a workpiece that can be exposed to a process includes a first sensor site for receiving a first sensor and at least one second sensor site for receiving a second sensor, so that the first measured data can be captured with the first sensor and second measured data with the second sensor. The second sensor site is designed and disposed in the multi-sensor head such that it can be equipped with sensors of different types for performing a measurement. The multi-sensor head can be part of a measurement system using two exchangeable sensors of different types in the multi-sensor head. The multi-sensor head can be part of a workpiece.

### TECHNICAL FIELD

The invention relates to a multi-sensor head for installation into an installation opening of a workpiece which can be exposed to a process, a measuring system with a multi-sensor head, the use of two or a plurality of sensors of various types in a multi-sensor head, and also a workpiece with a process chamber equipped with a multi-sensor head according to the invention.

### PRIOR ART

A multiplicity of different sensors for measuring a large range of measurement variables are known in the prior art. Sensors, which for example detect temperature, heat flow, acceleration, light, travel, deformation, pressure or other data of machines or tools in order to analyse and to monitor certain operating states or in order to control or to regulate these apparatuses, are becoming more and more important.

Often it is important to measure a plurality of interesting measurement variables at the same time or at the same location to the greatest extent possible. Combination sensors are therefore known, in which for example two or a plurality of elementary sensors are fixedly mounted in a common housing. A combination sensor of this type is for example disclosed in AT 505 077 A2, which comprises a pressure sensor which can be inserted into a hole which opens into a combustion chamber of an internal combustion engine, the pressure sensor having an optical waveguide optically opening into the combustion chamber, in the case of which an optical coupling element is provided in the opening region in the combustion chamber. An optical measurement and a pressure measurement are therefore possible at the same time in the smallest possible space with the combination sensor of AT 505 077 A2. In this case, such combination sensors are also very well known in the prior art in other embodiments. For example, in addition to an actually interesting measurement signal, the temperature may also be of importance, because the actually interesting measurement signal is influenced by the temperature and the temperature signal is therefore needed for correction. For such objects, combination sensors are known, which additionally comprise a temperature sensor, for example in the form of a thermocouple.

A device is known from US 2003/0145646, which can accommodate sensor elements of a predefined type at certain locations which are correspondingly prepared and already provided with appropriate connection lines. These sensor elements cannot be used by themselves however, as they lack the connection lines. It is additionally disadvantageous that they have to be dismantled in the case of exchange.

A further combination sensor, which has a plurality of fixedly installed sensor elements, is known from EP 1415698.

Often, however, the object is that a plurality of measurement variables must be measured at the same time on one measurement object, for example in a combustion chamber of an internal combustion engine, a turbine or for example on an injection moulding machine or an injection mould. Often, then there are not enough mounting positions available or correspondingly many mounting positions must be created with a large amount of outlay. If this is not possible, the necessary measurements must be undertaken temporally one after the other or expensive combination sensors must be used, in which various types of sensors are fixedly mounted at the same time.

In practice, it is often the case that a central measurement variable, such as for example the pressure in the combustion chamber of an internal combustion engine, must be determined in combination with a plurality of other variables, for example in combination with the temperature or a heat flow, or the pressure in combination with an expansion measurement or an optical measurement, etc. Then, a corresponding set of various combination sensors must be made available, of which each for example comprises a pressure sensor and one further additional sensor for measuring a further interesting measurement variable in each case.

To determine a complete set of interesting measurement variables the various combination sensors must therefore be mounted and demounted again one after the other in a complex procedure or a whole range of mounting positions must be provided if more variables must be detected at the same time than a combi-sensor can detect, so that a plurality of combination sensors can be installed at the same time, which is very space-intensive, time consuming and inflexible in terms of handling and therefore ultimately leads to increasing of the costs.

### DESCRIPTION OF THE INVENTION

It is therefore the object of the invention to provide a novel multi-sensor head which avoids the disadvantages known from the prior art and enables the simultaneous measurement of a plurality of phenomena at one and the same mounting location in different sensor combinations, without the multi-sensor head having to be exchanged in an expensive manner during the change to a sensor of another type.

The subject matters of the invention achieving these objects are characterised by the features of the independent claims.

The dependent claims relate to particularly advantageous embodiments of the invention.

The invention thus relates to a multi-sensor head for installation into an installation opening of a workpiece which can be exposed to a process, the multi-sensor head comprising a first sensor space for accommodating a first sensor and at least one second sensor space for accommodating a second sensor, so that first measurement data can be detected with the first sensor and second measurement data can be detected with the second sensor. According to the invention, the second sensor space is configured and arranged in the multi-sensor head in such a manner that for carrying out a measurement it can be equipped in an exchangeable manner with sensors of a different type. The invention furthermore relates to a measuring system with a multi-sensor head, the use of two or a plurality of exchangeable sensors of various types in a multi-sensor head, and also a workpiece with a multi-sensor head of the present invention.

By means of the multi-sensor head of the present invention, it is therefore first possible to realise a plurality of different measurement variables at one and the same mounting location in different sensor combinations without the multi-sensor head having to be exchanged in an expensive manner during the change to a sensor of another type. This is achieved according to the invention in that the multi-sensor head comprises at least one sensor space which is configured and provided in the multi-sensor head in such a manner that for carrying out different measurements it can be equipped in an exchangeable manner with sensors of a different type.

Thus, when changing to another sensor type, the multi-sensor head according to the invention does not need to be expensively demounted and exchanged with another sensor head which comprises the required sensor, rather the no longer required sensor can simply be removed from its sensor space of the multi-sensor head and exchanged with another sensor of another type which is required for the planned new measurement, without having to demount the multi-sensor head in the process.

So that this is possible, it must be possible to install complete sensors incl. their specific connection cables, as can be used by themselves. A connection cable of a thermocouple naturally has different requirements on the connections, lines and insulations than a piezoelectric measuring element for example. Thus, the multi-sensor head is [lacuna] via no connection wiring of the exchangeable sensors.

The most important characteristic of the multi-sensor is that it enables as many measurements as possible and in flexible combinations with as few holes as possible into the process chamber.

The invention furthermore relates to the use of two or a plurality of sensors of various types in a multi-sensor head of the present invention, the sensors of various types being configured in such a manner that they can be placed in an exchangeable manner in one and the same sensor space of the multi-sensor head.

Furthermore, the invention also relates to a measuring system for measuring a measurement variable in a workpiece, comprising a multi-sensor head according to the invention, and also a sensor which is suitable for installation in the multi-sensor head.

Finally, the invention also comprises a workpiece which can be exposed to a process, which in particular comprises a process chamber, the process chamber in particular being a heat engine, an internal combustion piston engine, a rotary piston engine, a gas turbine, an aircraft engine, an injection moulding machine or an injection mould with a multi-sensor head of the present invention installed in an installation opening of the workpiece.

### SHORT DESCRIPTION OF THE DRAWINGS

In the following, the invention is explained in more detail on the basis of the drawing. In the figures:

FIG. 1 shows a multi-sensor head according to the invention for accommodating two exchangeable sensors in a schematic illustration;

FIG. 2 shows an exemplary embodiment with a non-exchangeable basic sensor in a schematic illustration;

FIG. 3 shows a further exemplary embodiment according to FIG. 2 in a schematic illustration;

FIG. 4 shows an exemplary embodiment according to FIG. 3 with an installation-space-optimised basic sensor in a schematic illustration;

FIG. 5 shows a second exemplary embodiment according to FIG. 2 with a central free space in the basic sensor in a schematic illustration;

FIG. 6 shows a two-part multi-sensor head with basic-part and add-on part in a schematic illustration;

FIG. 7 shows a second exemplary embodiment according to FIG. 6 in a schematic illustration;

FIG. 8 shows a triple multi-sensor head with an auxiliary sensor in the basic sensor in a schematic illustration.

### WAYS OF REALISING THE INVENTION

FIG. 1 shows a first simple exemplary embodiment of a multi-sensor head according to the invention for accommodating two exchangeable sensors in a schematic illustration, the multi-sensor head according to the invention being designated with the reference symbol 1 as a whole in the following.

In the specific exemplary embodiment of FIG. 1, the multi-sensor head 1 is installed in a cylinder head 3 of an internal combustion engine in an installation opening 2 in order to carry out different measurements in the combustion chamber 31. The multi-sensor head 1 in this case comprises two sensor spaces 4, 5 which are both configured in the form of a hole in such a manner that each of the two sensor spaces can be equipped in an exchangeable manner with sensors 41, 51, 52 of a different type for carrying out different measurements. In this case, the hole preferably comprises a screw connection which is not illustrated explicitly in FIG. 1, so that the exchangeable sensor 41, 51, 52 can be screwed in the hole easily.

In this case, the sensors of a different type can in each case measure a different physical measurement variable and/or have a different measurement principle and/or have a different sensitivity or a different measuring range.

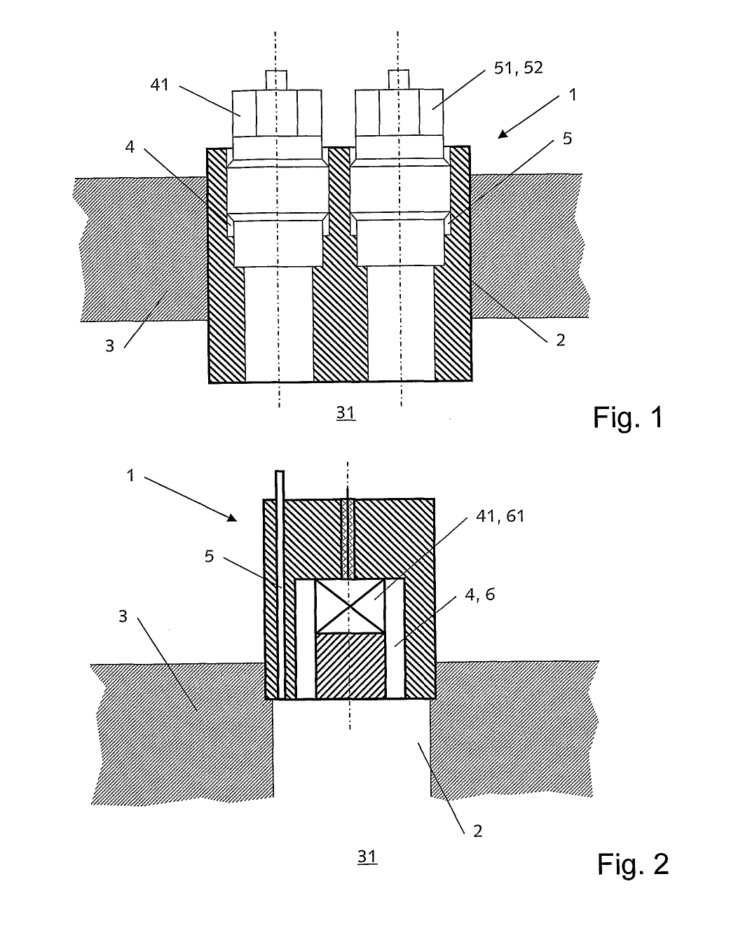

In FIG. 2, an exemplary embodiment with a centrally arranged first sensor 41 is illustrated schematically, whereby the first sensor 41 is not a non-exchangeable basic sensor 61 here and a second sensor space 5 is arranged asymmetrically with respect to a central axis of the multi-sensor head 1 in the cover of the basic sensor head 1. The non-exchangeable basic sensor 61 is a pressure sensor here, whereby the second sensor space 5 is not yet equipped with a sensor 51, 52, e.g. a thermocouple for surface temperature measurement. A multi-sensor head 1 according to FIG. 2 can be used advantageously then in particular if a basic variable, such as e.g. the pressure in a process chamber 31 of the workpiece 3 must be measured at the same time as a plurality of other variables one after the other. So, e.g. in a first measurement, the pressure and the temperature are measured at the same time in the process chamber 31. For a further measurement, the temperature sensor 51, 52, which is provided in an exchangeable manner in the sensor space 5, is then exchanged with a sensor 51, 52 of another type, in order to measure another variable at the same time as the pressure in the further measurement.

The sensor 41, 51, 52, 61 can in this case for example be a sensor for measuring a heat flow, or a temperature, or an acceleration, or an optical signal, or a sensor for determining the upper dead-centre position in a piston engine, or a sensor for measuring a distance, or a deformation, or a force, or a pressure.

The sensor 41, 51, 52, 61 is in this case in particular a piezoelectric sensor, a piezoresistive sensor, a sensor with a thermocouple or a temperature-dependent electrical resistance or a semiconductor diode, or the sensor 41, 51, 52, 61 is a frequency-analogue sensor, a capacitive sensor, an inductive sensor, an optical sensor, a mechanical sensor, a sensor with a strain gauge or a sensor with a Hall element.

The process chamber 31 of the workpiece 3 is in particular a combustion chamber of a heat engine, particularly a cylinder of an internal combustion piston engine, or a combustion chamber of a rotary piston engine, or a process chamber 31 of a turbine, particularly a gas turbine or an aircraft engine, or the process chamber 31 is a chamber of an injection moulding machine or an injection mould.

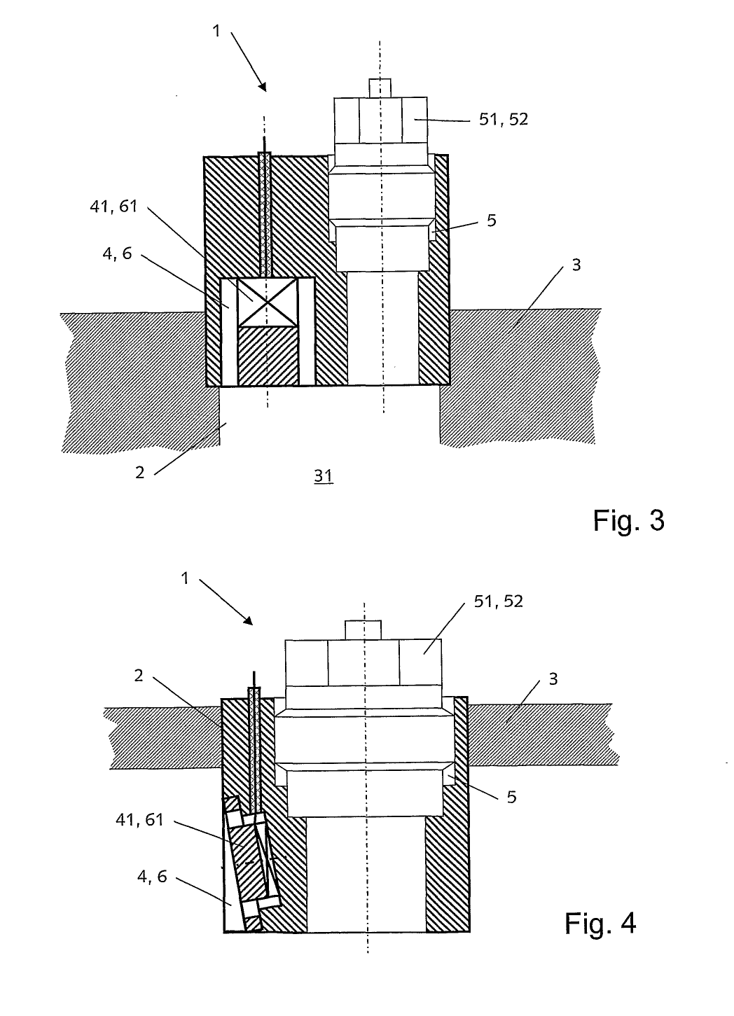

FIG. 3 shows a further exemplary embodiment according to FIG. 2 with a first sensor space 4, in which a first sensor 41 is provided as fixedly installed basic sensor 61, wherein here, by contrast with FIG. 2, a relatively large second sensor 51, 52 is installed in the second sensor space 5 here and the basic sensor space 6 and the second sensor space 5 are arranged essentially symmetrically to the central axis of the multi-sensor head 1.

FIG. 4 shows an exemplary embodiment according to FIG. 3 with an installation-space-optimised first sensor 41 which is provided as a fixedly installed basic sensor 61 in the basic sensor space 6 which is here, as in FIG. 3, identical to the first sensor space 4. According to the invention, a second sensor space 5 is also provided here for accommodating a second sensor 51, 52. Here, the basic sensor 61 is fixedly installed in an extremely space-saving manner in the multi-sensor head 1, so that sufficient installation space remains for the exchangeable sensors 51, 52.

Incidentally, it is understood that in a multi-sensor head 1 according to the invention more than one second sensor space 5 can also be provided for accommodating exchangeable sensors 51, 52, as a result of which the multi-sensor head 1 according to the invention becomes even more flexible and simpler in terms of handling, because more than two measurement variables can be measured at the same time in the process chamber 31.

In this case, the first sensor space 4 and/or the second sensor space 5 and/or the basic sensor space 6 can also particularly be configured and arranged in the multi-sensor head in such a manner that two or more sensors of the same or different type can be placed in the first sensor space 4 and/or in the second sensor space 5 and/or in the basic sensor space 6 at the same time.

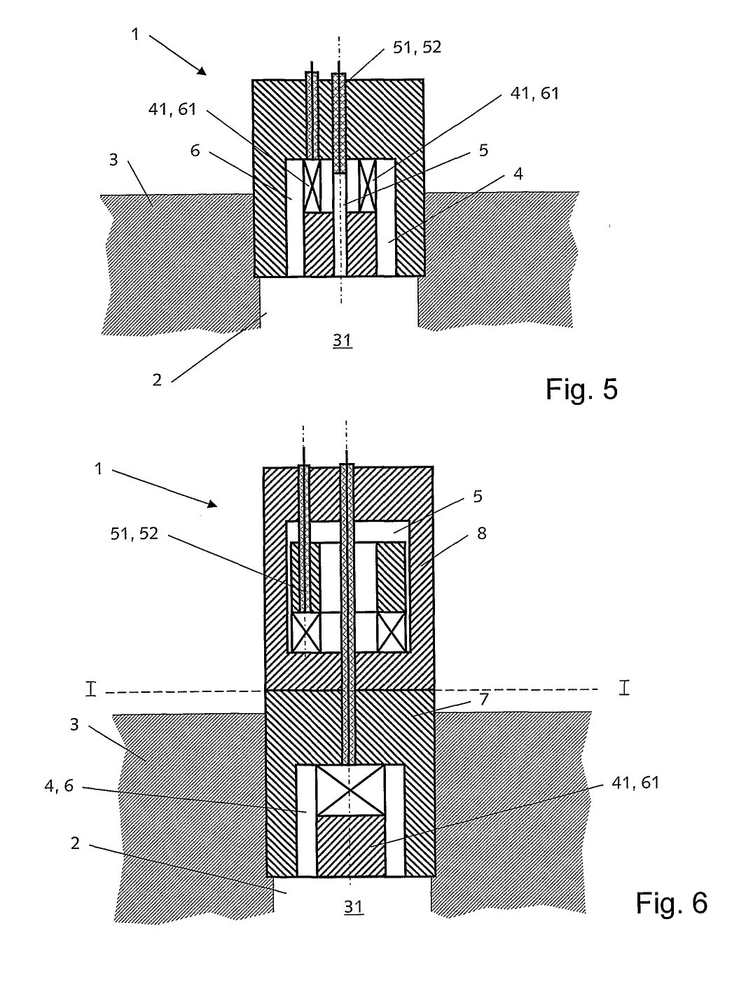

In FIG. 5, a second exemplary embodiment according to FIG. 4 is illustrated with a second sensor space 5 configured as a central free space 5 in the basic sensor 61, in which central free space 5 a heat flow for example particularly advantageously can be measured with an exchangeable heat flow sensor 51, 52 in the process chamber 31, whilst a basic variable, such as the pressure or the temperature can be monitored in the process chamber 31 at the same time for example with the fixedly installed sensor 41, 61. If, for another application, not the heat flow, but rather for example a temperature is required, then the heat flow sensor 51, 52 can easily be exchanged with a temperature sensor 51, 52.

For special applications, a two-part multi-sensor head 1 with a basic part 7 and an add-on part 8, which are assembled in a separable manner at a separation point I-I, are schematically illustrated in FIG. 6. Here, the exchangeable sensor 51, 52 is therefore not screwed in a hole, as has been explained on the basis of FIG. 1 by way of example. Rather, the exchange of the sensor 51, 52 can take place such that the add-on part 8, in which a sensor of a certain type is provided, is exchanged with another add-on part 8 which contains a sensor of another type. It is understood that the sensor 51, 52 can of course also be provided in an exchangeable manner in the add-on part 8, so that when changing to another sensor 51, 52, the entire add-on part 8 is not exchanged, but rather only the sensor 51, 52 in the add-on part 8 is exchanged.

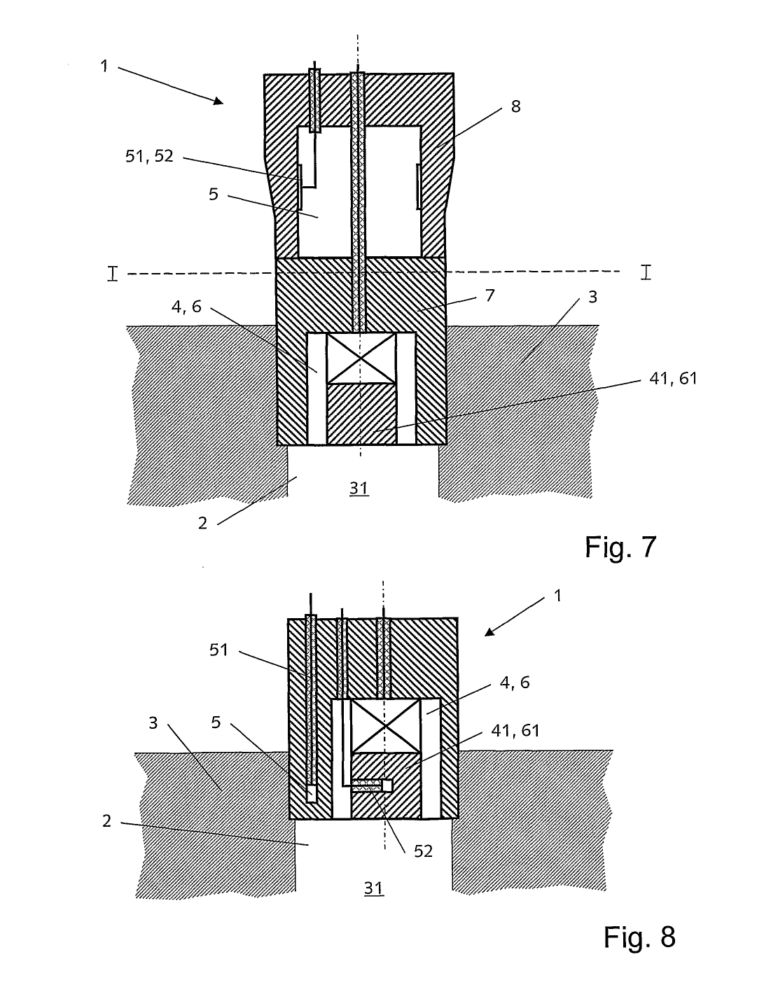

In FIG. 7, a second exemplary embodiment according to FIG. 6 is shown, in which a deformation sensor 51, 52 is provided in the add-on part 8, which is placed on the basic part 7 at the separation point I-I, and in the basic part 7 a first sensor is provided in a first sensor space 4, whereby the first sensor 41 can either be an exchangeable sensor or a non-exchangeable basic sensor 61.

Finally, a triple multi-sensor head 1 is also explained on the basis of FIG. 8, which in addition to the second sensor 51 also has a third auxiliary sensor 52 in the first sensor 41, which in the present example is realised as a non-exchangeable basic sensor 61. This is a particularly compact design here, as the exchangeable sensor 52 is provided within the first sensor 41 and therefore takes up practically no additional space.

A further advantage of the multi-sensor head 1 consists in the fact that, in a preferred embodiment, one or a plurality of the sensors 41, 51, 52 can be exchanged, whilst the multi-sensor head 1 remains installed in the installation opening 2. This is particularly advantageous if the removal of the multi-sensor head 1 is complicated. Particular outlay results for example if the multi-sensor head 1 must be orientated in a very planar manner with respect to a mould wall, seals a process chamber 31 which is pressurised or filled with a liquid, or if it must be installed from its front side which is only accessible following a great deal of demounting outlay. An exchangeability of one or a plurality of sensors 41, 51, 52 in the installed state of the multi-sensor head 1 then saves a great deal of work.

In this case, one or a plurality of sensor spaces 4, 5 are preferably configured and arranged in the multi-sensor head in such a manner that an exchange of one or a plurality of sensors 41, 51, 52 can be carried out in the installed state of the multi-sensor head 1 in a workpiece 3.

### REFERENCE LIST

1 Multi-sensor head
2 Installation opening
3 Workpiece
31 Process chamber
4 First sensor space
41 First sensor
5 Second sensor space
51, 52 Second sensor
6 Basic sensor space
61 Basic sensor
7 Basic part
8 Add-on part
I-I Separation point

1. A multi-sensor head for installation into an installation opening of a workpiece which can be exposed to a process, wherein the multi-sensor head comprises a first sensor space for accommodating a first sensor and at least one second sensor space for accommodating a second sensor, so that first measurement data can be detected with the first sensor and second measurement data can be detected with the second sensor, wherein the second sensor space is configured and arranged in the multi-sensor head in such a manner that for carrying out a measurement it can be equipped in an exchangeable manner with exchangeable sensors of a different type.

2. The multi-sensor head according to claim 1, wherein the exchangeable sensors of a different type can in each case measure a different physical variable and/or have a different measurement principle and/or have a different sensitivity and/or have a different measuring range.

3. The multi-sensor head according to claim 1, wherein the first sensor and the second sensor are of the same type.

4. The multi-sensor head according to claim 1, wherein the first sensor space is configured and arranged in the multi-sensor head in such a manner that for carrying out a measurement it can be equipped in an exchangeable manner with exchangeable sensors of a different type.

5. The multi-sensor head according to claim 1, wherein the multi-sensor head further comprises a basic sensor space with a non-exchangeable basic sensor for measuring a basic measurement variable.

6. The multi-sensor head according to claim 5, wherein the first sensor space and/or the second sensor space and/or the basic sensor space are configured and arranged in the multi-sensor head in such a manner that two sensors of the same or different type can be placed in the first sensor space and/or in the second sensor space and/or in the basic sensor space at the same time.

7. The multi-sensor head according to claim 1, wherein the multi-sensor head is configured in a two-part manner and comprises an add-on part which can be separated from a basic part at a separation point.

8. The multi-sensor head according to claim 5, wherein the first sensor space and/or the second sensor space and/or the basic sensor space comprises a hole for accommodating the exchangeable sensor.

9. The multi-sensor head according to claim 8, wherein a screw connection is provided at the hole, so that the exchangeable sensor can be screwed into the hole.

10. The multi-sensor head according to claim 1, wherein each exchangeable sensor is a sensor for measuring a heat flow, or a temperature, or an acceleration, or an optical signal, or a sensor for determining the upper dead-centre position in a piston engine, or a sensor for measuring a distance, or a deformation, or a force, or a pressure.

11. The multi-sensor head according to claim 1, wherein each exchangeable sensor is a piezoelectric sensor, or a piezoresistive sensor, or a sensor with a thermocouple or a temperature-dependent electrical resistance or a semiconductor diode, or wherein each exchangeable sensor is a frequency-analogue sensor, or a capacitive sensor, or an inductive sensor, or an optical sensor, or a mechanical sensor, or a sensor with a strain gauge or a sensor with a Hall element.

12. The multi-sensor head according to claim 1, wherein the workpiece in particular comprises a cylinder of an internal combustion piston engine, or a combustion chamber of a rotary piston engine, or a process chamber of a gas turbine or an aircraft engine, or wherein the process chamber is a chamber of an injection moulding machine or an injection mould.

13. The multi-sensor head according to claim 1, wherein at least one of sensor spaces is configured and arranged in the multi-sensor head in such a manner that an exchange of at least one of the sensors can be carried out in the installed state of the multi-sensor head in a workpiece.

14. Use of two sensors of various types in a multi-sensor head according to claim 1, wherein the sensors are configured in such a manner that they can be placed in an exchangeable manner in one and the same sensor space of the multi-sensor head.

15. A measuring system for measuring a measurement variable in a workpiece, comprising a multi-sensor head according to claim 1, and also a sensor for installation in the multi-sensor head.

16. A workpiece which can be exposed to a process, comprising a process chamber, wherein the process chamber in particular is a heat engine, or an internal combustion piston engine, or a rotary piston engine, or a gas turbine, or an aircraft engine, or an injection moulding machine or an injection mould with a multi-sensor head according to claim 1 installed in an installation opening of the workpiece.

17. The multi-sensor head according to claim 1, wherein the first sensor space and/or the second sensor space are configured and arranged in the multi-sensor head in such a manner that two sensors of the same or different type can be placed in the first sensor space and/or in the second sensor space at the same time.

18. The multi-sensor head according to claim 1, wherein the first sensor space and/or the second sensor space comprises a hole for accommodating the exchangeable sensor.

19. The multi-sensor head according to claim 18, wherein a screw connection is provided at the hole, so that the exchangeable sensor can be screwed into the hole.
